(19) United States
(12) Patent Application Publication
(10) Pub. No.: US 2025/0134095 A1
(43) Pub. Date: May 1, 2025

(54) COMPOSITIONS FOR ORGAN AND TISSUE PRESERVATION

(71) Applicants: Albert Einstein College of Medicine, Bronx, NY (US); Yale University, New Haven, CT (US)

(72) Inventors: Evripidis Gavathiotis, Roslyn, NY (US); Richard N. Kitsis, Bronx, NY (US); Gregory Tietjen, New Haven, CT (US); Jenna Dirito, New Haven, CT (US)

(21) Appl. No.: 18/683,106

(22) PCT Filed: Aug. 19, 2022
(86) PCT No.: PCT/US2022/040874

§ 371 Date: Feb. 12, 2024

Related U.S. Application Data
(60) Provisional application No. 63/235,455, filed on Aug. 20, 2021.

(51) Int. Cl. A01N 1/126; C12N 5/00

(52) U.S. Cl. CPC A01N 1/126; C12N 5/526

(57) ABSTRACT

Disclosed are compositions and methods for the preservation, storage, and transport of living biological tissues, organs, and populations of isolated cells. In particular, the compositions and methods permit mammalian cells, tissues, and organs to be recovered from suitable donor animals, stored for prolonged periods, and transported to the site of recipient implantation, all without significant loss of cell viability, biological activity, and/or tissue integrity.

### CROSS-REFERENCE AND RELATED APPLICATION

This patent document is a national phase filing of International Application No. PCT/US22/40874 filed Aug. 19, 2022, which claims priority to U.S. Provisional Patent Application 63/235,455 filed on Aug. 20, 2021, the content and teachings of both of which are incorporated herein by reference in their entirety.

### STATEMENT REGARDING FEDERALLY SPONSORED RESEARCH OR DEVELOPMENT

This invention was made with government support under grant number DK128662 awarded by NIDDK NIH HHS/United States and under grant number DK124420 awarded by NIDDK NIH HHS/United States. The government has certain rights in the invention.

### TECHNICAL FIELD

The present application relates generally to the field of biological tissue preservation, storage, and transport.

### BACKGROUND

A significant limitation to meeting the annual worldwide need for tissue allografts and organs (including without limitation heart, lung, liver, kidney, spleen, stomach, intestine, pancreas, eye, bone, bone marrow, cochlea, testis, ovary and all other solid and blood-related organs and tissues) is the relative difficulty for controlling the delicate balance between the “supply” of viable explants from suitable donors and the “demand” of transplant candidates across the globe. Even in those circumstances in which suitable donor(s) and recipient(s) can be matched, another important limitation is the ability to store, screen, match, and transport tissues along the path from the site of donor harvest, to the site of the tissue storage repository, and then onward to the site of recipient transplant—a path that is logistically challenging in being highly time-sensitive and often involving thousands of miles. Unlike mammalian blood and blood components, which may be recovered and “banked” for several weeks without significant loss of viability, most mammalian tissues and organs, in contrast, are quite fragile. For example, the post-recovery time interval during which many human tissues remain viable (even if stored and transported under currently ideal conditions) is typically less than a day and often only a few hours. Similarly, most mammalian organs rapidly lose viability and function after removal from the donor, and may become unsuitable for transplantation after extracorporeal storage and transport as soon as six to eight hours post-harvest.

Even for mammalian tissues that are most amenable to post-harvest tissue banking, the critical “window of opportunity” between harvest and transplant is only a few weeks at best. As a result, often there is not enough time to match donors and recipients, test the quality and suitability of the explant, transport the tissue from the donor to the recipient, and implant the tissue into the recipient. Consequently, there are substantially more recipients awaiting transplants than there are suitable donor tissues available for transplant.

The fact that conventional buffer solutions, physiological formulations, diluents, standard culture media, cellular growth media, tissue storage solutions, and organ transport media are typically only able to preserve the cellular viability and suitability of biological tissues or organs for transplantation for a period of a few hours to a few days post-harvest makes them largely unsuitable for prolonged- or extended-term storage of viable biological materials such as mammalian cells, tissues, organs, explants, and the like. Therefore, a need exists for compositions and methodologies that facilitate the long-term preservation of cell, tissue, and organ viability, and that maintain biological activity, function, and tissue integrity.

### SUMMARY OF THE INVENTION

Various embodiments of this patent document provide cell-, tissue-, and organ-preserving compositions and methods for the collection, analysis, screening, storage, transport, and transplantation of such recovered biological materials into a recipient animal. In comparison to existing preservation solutions, or biological buffers or media alone, the compositions and methods improve cellular, tissue and organ viability and provide enhanced preservation capabilities.

An aspect of the patent document provides a method of reducing cell death in a biological material ex vivo. The method includes contacting the biological sample with an effective amount of a compound of Formula I or a pharmaceutically acceptable salt thereof. By maintaining the contact between the sample and the compound, the sample remains viable (e.g. more than 30%, more than 50%, more than 80%, or more than 95% of cell population in the sample) during the period of contact.

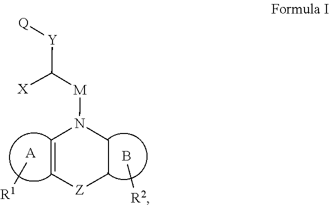

wherein
A is phenyl or a 6-membered heteroaromatic ring having 1, 2 or 3 N atoms in the heteroaromatic ring;
B is phenyl, or a 6-membered heteroaromatic ring having 1, 2 or 3 heteroatoms (N, O and/or S) in the heteroaromatic ring, or a 6-membered aliphatic ring wherein optionally 1, 2 or 3 carbons of the aliphatic ring are replaced with heteroatoms (N, O and/or S);
Z is void, a bond, O, S, NR^{a}, C(R^{a})₂, S(O)₂, or C(O);
the dashed line between A and B indicates an optional bond;
R¹ and R² are independently H, C₁₋₆ alkyl, F, Cl, Br, I, CN, NO₂, N(R^{a})₂, OC₁₋₆ alkyl, CF₃, COOH, COOC₁₋₆ alkyl, NHC₁₋₆ alkyl, OC(C═O) C₁₋₆ alkyl, OC₁₋₆ alkyl, (C═O) C₁₋₆ alkyl, C₁₋₆ alkylene-OH, SC₁₋₆ alkyl, SOC₁₋₆ alkyl, or SO₂C₁₋₆ alkyl;
X is H, NH₂, OH, F, Cl, Br, I, CN, SH, NO₂, N(R^{a})₂, OR^{a}, CF₃, COOH, C₁₋₆ alkyl, COO C₁₋₆ alkyl, OCC₁₋₆ alkyl, OCO C₁₋₆ alkyl, O C₁₋₆ alkyl, S C₁₋₆ alkyl, SO C₁₋₆ alkyl, or SO₂ C₁₋₆ alkyl;
M is C₁₋₆ alkylene;
Y is a bond or C₁₋₆ alkylene;
Q is OH, COOH, or N(R^{b})₂,
R^{a} is H or C₁₋₆ alkyl;
R^{b} in each instance is independently H, C₁₋₆ alkyl, or C₂₋₆ alkyleneR^{c},
R^{c} is OH, SH, N(R^{a})₂, NR^{a}C₂₋₆ alkylene-OH, or N(C₂₋₆ alkylene-OH)₂,
   wherein two R^{b} optionally link up to form a ring.

The method is applicable to collection, analysis, screening, storage, transport, and transplantation of biological materials including cells, tissues, and organs. In some embodiments, the method enhances viability of the biological materials during cold storage either with or without a preceding period of warm ischemic injury.

Also provided is a kit including the composition disclosed herein for collection, analysis, screening, storage, transport, and transplantation of biological materials.

### DESCRIPTION OF DRAWINGS

FIGS. 1(A)-1(D) shows cell death in human, pig, and mouse kidneys during extended periods of cold preservation. FIG. 1(A) shows that cell death arises in human, pig, and mouse kidneys during extended periods of cold preservation. FIG. 1(B) shows the change in positive cells over cold ischemic time for human kidney. FIG. 1(C) shows the change in positive cells over cold ischemic time for mouse kidney. FIG. 1(D) shows the change in positive cells over cold ischemic time for pig kidney.

FIG. 2 shows that cell death arises in mouse concurrent with Bax activation during prolonged cold storage.

FIGS. 3(A)-3(E) shows the effect of genetic or pharmacological BAX inhibition on cell death during prolonged cold storage of mouse kidneys. FIG. 3(A) shows genetic or pharmacological BAX inhibition abrogates cell death during prolonged cold storage of mouse kidneys. FIG. 3(B) shows the change in positive (dead) cells over cold ischemic time for control. FIG. 3(C) shows the change in positive cells over cold ischemic time for Bax knockout mice. FIG. 3(D) shows the change in positive cells over cold ischemic time for mouse kidney, where BAI1 was given intraperitoneally to the animal 24 hours before the kidney recovery then BAI1 was also included in the preservative solution. FIG. 3(E) shows the change in positive cells over cold ischemic time for mouse kidney using BAI1 supplemented cold preservation solution. BAI1 was only included in the preservative solution.

FIG. 4 shows example compounds of BAX inhibitors.

FIG. 5 shows example compounds of BAX inhibitors.

FIG. 6 shows example compounds of BAX inhibitors.

### DETAILED DESCRIPTION

Various embodiments of this patent document disclose compositions and methods for tissue and organ storage and preservation. By inhibiting BAX-mediated cell death, the compositions and methods provide a significant improvement in long-term viability and suitability of such biological materials for periods of from several weeks to several months or more. These methods substantially increase the time interval during which explanted biological material remains viable and suitable for transplantation, and thus lengthen the critical window between the initial harvesting of tissues/organs and their subsequent implantation in a suitable recipient.

The articles “a” and “an” as used herein refer to “one or more” or “at least one,” unless otherwise indicated. That is, reference to any element or component of an embodiment by the indefinite article “a” or “an” does not exclude the possibility that more than one element or component is present.

The term “about” as used herein generally refers to plus or minus 10% of the indicated number. For example, “about 10%” may indicate a range of 9% to 11%, and “about 20” may mean from 18 to 22. Other meanings of “about” may be apparent from the context, such as rounding off, so, for example “about 1” may also mean from 0.5 to 1.4. As used herein, the term “and/or” includes any and all combinations of one or more of the associated listed items.

The term “alkyl” refers to a hydrocarbon or a hydrocarbon chain which may be either straight-chained or branched. The term “C₁₋₆ alkyl” refers to alkyl groups having 1, 2, 3, 4, 5 or 6 carbon atoms. Non-limiting examples include groups such as CH₃, (CH₂)₂CH₃, CH₂CH(CH₃)CH₃, and the like. Similarly, the term “C₂₋₅ alkyl” refers to alkyl groups having 2, 3, 4 or 5 carbon atoms.

The term “alkylene” refers to a methylene or a hydrocarbon chain which may be either straight-chained or branched. Different from alkyl which has only one point of bonding with other groups or atoms, alkylene has two points of bonding. Non-limiting examples include groups such as CH₂, (CH₂)₂, CH₂CH(CH₃), and the like. A C₂₋₆alkylene has 2, 3, 4, 5 or 6 carbons.

The term “biological material” refers to a material that is obtained from a biological entity, or any population of cells and/or tissues that are of biological origin. Such sources include, without limitation, whole or dissected tissues, including cells, tissues or organs obtained from biopsy, autopsy, and/or necropsy, as well as aspirates or lavages; in situ or in vitro cells (including, e.g., individual cells, populations of cells, confluent or monolayer cells, or cell populations, transformed cell lines, tissue and/or cellular explants, tissue-engineered constructs (TECs), tissue-engineered devices (i.e., TEDs), tissue engineered products (TEPs) in vivo, in vitro, in situ, ex situ, and ex vivo biological grafts, allografts, autografts, isografts, xenografts, autologous graft tissues, as well as muscle-tendon grafts, structural spine units, and such like.

The term “ex vivo” in the context of a biological material refers to the biological material being outside of the living body.

The terms “patient” and “recipient” are intended to include animals, and in particular, mammalian species such as human beings, livestock, or animals under veterinary care and/or supervision.

The term “tissue” refers to any biological material that is recovered from one animal and implanted into the same species of animal (allograft) or another species of animal (xenograft). The tissue may be from a whole or partial organ, such as a heart valve or an aorta, or from a specific location in the animal, such as cartilage or a tendon from the knee joint.

The terms “viable cell(s),” “viable tissue(s),” and “viable organ(s)” refers to one or more cells, tissues, and/or organs, respectively, that comprise at least a first population of living cells that are capable of surviving and substantially maintaining their extant biological function provided that they are harvested, stored, maintained, cultured, transported, and/or transplanted under the necessary biological conditions (e.g., nutrients, incubation temperature, etc.) effective to maintain the viability of such cells, tissues or organs sufficient for implantation into a suitable recipient host.

The term “effective amount” as used herein means that amount of an agent that will maintain the viability of the biological sample (e.g. cell, tissue, or organ).

The term “pharmaceutically acceptable salts” means salts of compounds of the present invention which are pharmaceutically acceptable, as defined above, and which possess the desired pharmacological activity. Non-limiting examples of such salts include acid addition salts formed with inorganic acids such as hydrochloric acid, hydrobromic acid, sulfuric acid, nitric acid, and phosphoric acid; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, and trimethylacetic acid. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Non-limiting examples of acceptable organic bases include ethanolamine, diethanolamine, triethanolamine, tromethamine, and N-methylglucamine. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The compositions and methods disclosed herein for the storage and/or transport of such biological samples have been shown to extend significantly the “shelf-life” of recovered donor cells, tissues, or organs. The compositions and methods facilitate intermediate- and/or long-term storage (i.e., banking) of a variety of biological samples under conditions suitable for maintaining the viability, integrity, or cellular activity of such samples in scenarios that are analogous to contemporary methodologies for the extended storage of viable blood and/or blood components that remain suitable for introduction into a suitable recipient organism.

### Composition

An aspect of this patent document provides a composition for preserving a cell, tissue, or organ for use in the collection, analysis, screening, storage, transport, and transplantation of such recovered biological materials into a recipient animal. In comparison to the use of existing preservation solutions, physiological fluids, diluents, or biological buffers or cell growth and/or culture media alone, these compositions exhibit superior performance and provide enhanced cellular preservation capabilities and enhanced maintenance of cellular viability.

The composition generally includes a compound of Formula I or a pharmaceutically acceptable salt thereof.

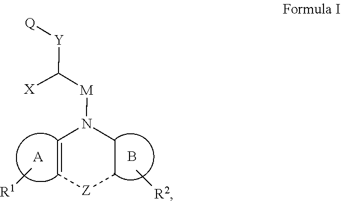

wherein
A is phenyl or a 6-membered heteroaromatic ring having 1, 2 or 3 N atoms in the heteroaromatic ring;
B is phenyl, or a 6-membered heteroaromatic ring having 1, 2 or 3 heteroatoms (N, O and/or S) in the heteroaromatic ring, or a 6-membered aliphatic ring wherein optionally 1, 2 or 3 carbons of the aliphatic ring are replaced with heteroatoms (N, O and/or S);
Z is void, a bond, O, S, NR^{a}, C(R^{a})₂, CHR^{a}, S(O)₂, C(Me)₂ or C(O);
the dashed line between A and B indicates an optional bond;
R¹ and R² are independently H, C₁₋₆ alkyl, F, Cl, Br, I, CN, NO₂, N(R^{a})₂, OC₁₋₆ alkyl, CF₃, COOH, COOC₁₋₆ alkyl, NHC₁₋₆ alkyl, OC(C═O) C₁₋₆ alkyl, OC₁₋₆ alkyl, (C═O) C₁₋₆ alkyl, C₁₋₆ alkylene-OH, SC₁₋₆ alkyl, SOC₁₋₆ alkyl, or SO₂C₁₋₆ alkyl;
X is H, NH₂, OH, F, Cl, Br, I, CN, SH, NO₂, N(R^{a})₂, OR^{a}, CF₃, COOH, C₁₋₆ alkyl, COO C₁₋₆ alkyl, OCC₁₋₆ alkyl, OCO C₁₋₆ alkyl, O C₁₋₆ alkyl, S C₁₋₆ alkyl, SO C₁₋₆ alkyl, or SO₂ C₁₋₆ alkyl;
M is C₁₋₆ alkylene;
Y is a bond or C₁₋₆ alkylene;
Q is OH, COOH, or N(R^{b})₂,
R^{a} is H or C₁₋₆ alkyl;
R^{b} in each instance is independently H, C₁₋₆ alkyl, or C₂₋₆ alkyleneR^{c},
R^{c} is OH, SH, N(R^{a})₂, NR^{a}C₂₋₆ alkylene-OH, or N(C₂₋₆ alkylene-OH)₂,
wherein two R^{b} optionally link up to form a ring.

Nonlimiting examples of ring A include phenyl, pyrimidinyl, and pyridinyl. Nonlimiting examples of ring B include phenyl, piperidinyl, piperazinyl, pyrimidinyl, pyridinyl and morpholinyl.

In some embodiments, ring A and ring B are each a phenyl ring.

In some embodiments, Z and the dotted line connecting Z to A and B are void or non-present.

In some embodiments, Z is a bond or a group/atom connecting ring A and ring B. In some embodiments, Z is a bond directly connecting ring A and ring B. In some embodiments, Z is O, S, NR^{a}, C(R^{a})₂, S(O)₂, or C(O) connecting ring A and ring B.

In some embodiments, the compound is of formula I-a,

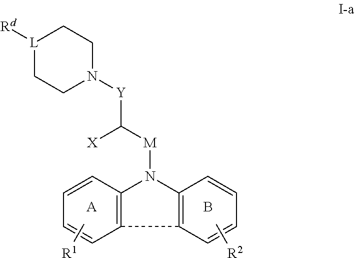

wherein L is O, S, N or CH; R^{d} is H, C₁₋₆ alkyl, or C₁₋₆ alkylene —OH, provided that when L is O or S, R^{d} is void to comply with the valency rule.

In some embodiments, the compound is of formula I-b,

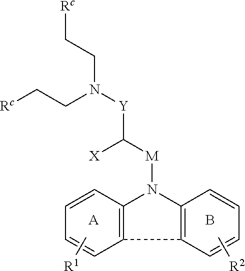

wherein R^{c} is OH, SH, NR^{a}C₂₋₆ alkylene-OH, or N(C₂₋₆ alkylene-OH)₂.

In some embodiments, the compound is of formula I-c,

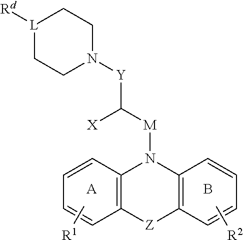

wherein Z is a bond, O, S, NR^{a}, C(R^{a})₂, S(O)₂, or C(O); L is O, S, N or CH; R^{d} is H, C₁₋₆ alkyl, or C₁₋₆ alkylene —OH)₂, provided that when L is O or S, R^{d} is void.

In some embodiments, the compound is of formula I-d,

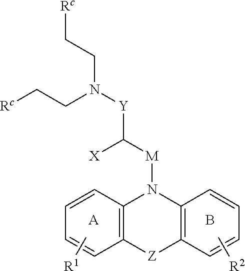

wherein R^{c} is OH, SH, NR^{a}C₂₋₆ alkylene-OH, or N(C₂₋₆ alkylene-OH)₂.

Nonlimiting examples of the compound of Formula I include the following:

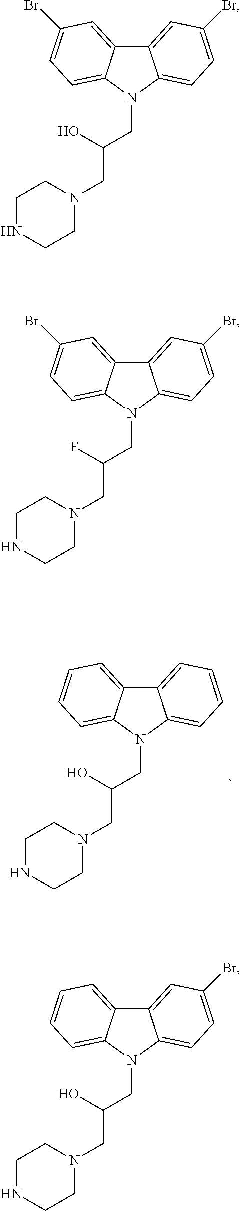 [SMILES: OC(CN1CCNCC1)Cn1c2ccc(Br)cc2c2cc(Br)ccc21 ; FC(CN1CCNCC1)Cn1c2ccc(Br)cc2c2cc(Br)ccc21 ; OC(CN1CCNCC1)Cn1c2ccccc2c2ccccc21 ; OC(CN1CCNCC1)Cn1c2ccccc2c2cc(Br)ccc21]

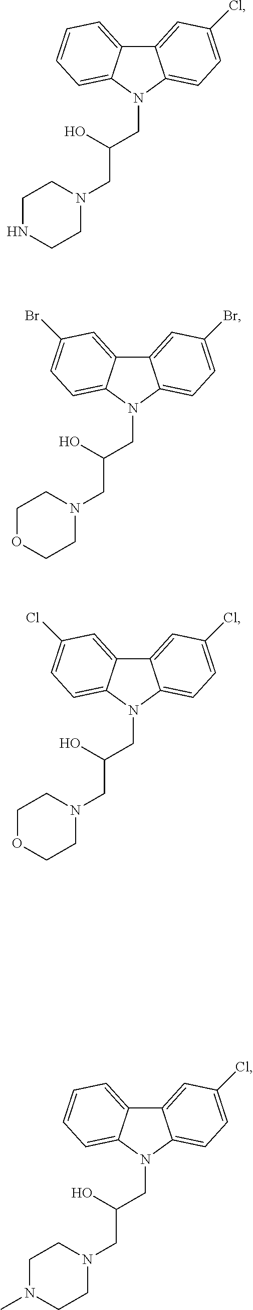 [SMILES: OC(CN1CCNCC1)Cn1c2ccccc2c2cc(Cl)ccc21 ; OC(CN1CCOCC1)Cn1c2ccc(Br)cc2c2cc(Br)ccc21 ; OC(CN1CCOCC1)Cn1c2ccc(Cl)cc2c2cc(Cl)ccc21 ; CN1CCN(CC(O)Cn2c3ccccc3c3cc(Cl)ccc32)CC1]

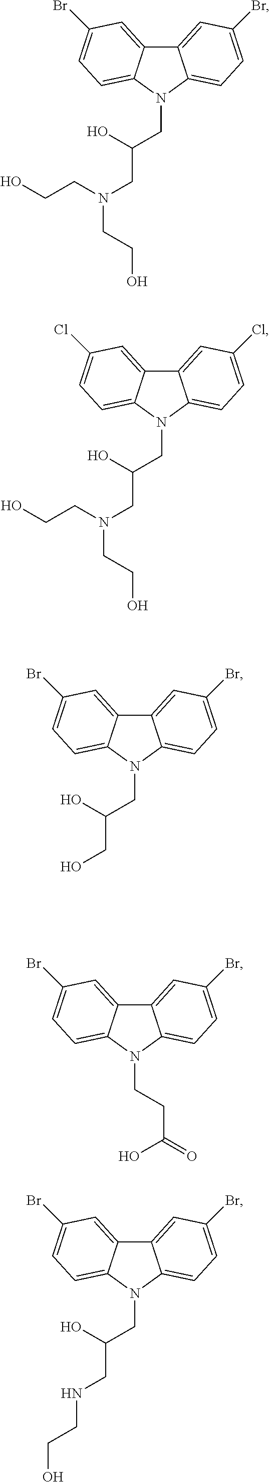 [SMILES: OCCN(CCO)CC(O)Cn1c2ccc(Br)cc2c2cc(Br)ccc21 ; OCCN(CCO)CC(O)Cn1c2ccc(Cl)cc2c2cc(Cl)ccc21 ; OCC(O)Cn1c2ccc(Br)cc2c2cc(Br)ccc21 ; O=C(O)CCn1c2ccc(Br)cc2c2cc(Br)ccc21 ; OCCNCC(O)Cn1c2ccc(Br)cc2c2cc(Br)ccc21]

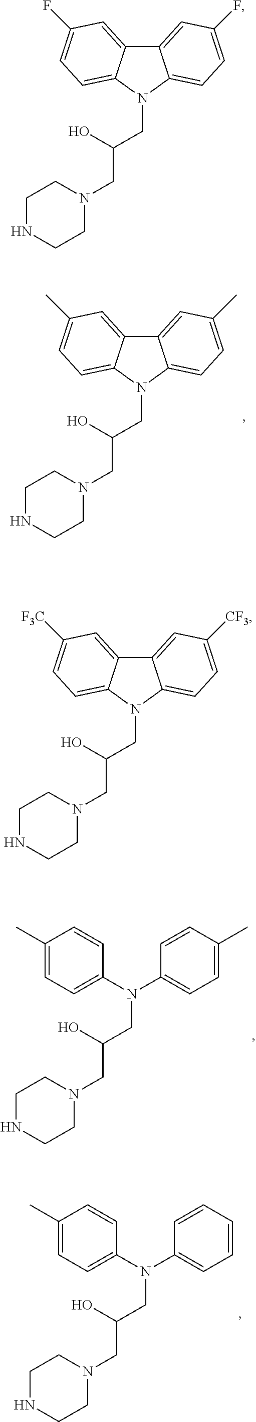 [SMILES: OC(CN1CCNCC1)Cn1c2ccc(F)cc2c2cc(F)ccc21 ; Cc1ccc2c(c1)c1cc(C)ccc1n2CC(O)CN1CCNCC1 ; OC(CN1CCNCC1)Cn1c2ccc(C(F)(F)F)cc2c2cc(C(F)(F)F)ccc21 ; Cc1ccc(N(CC(O)CN2CCNCC2)c2ccc(C)cc2)cc1 ; Cc1ccc(N(CC(O)CN2CCNCC2)c2ccccc2)cc1]

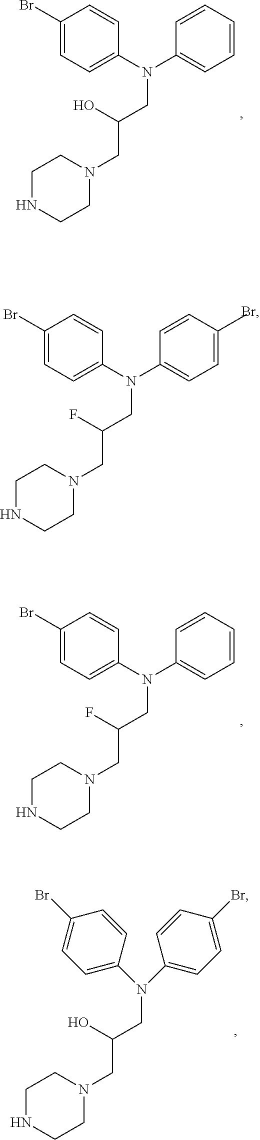

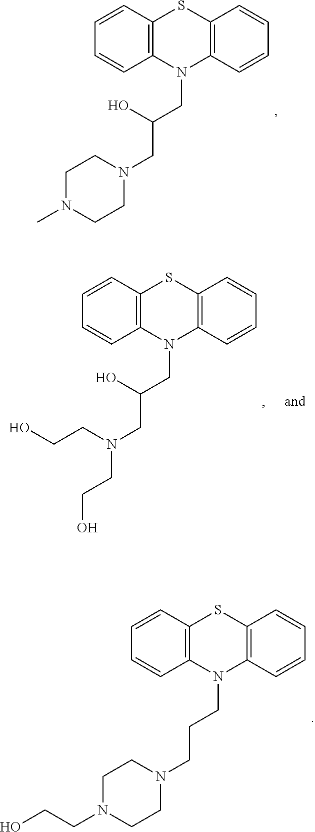 [SMILES: CN1CCN(CC(O)CN2c3ccccc3Sc3ccccc32)CC1 ; OCCN(CCO)CC(O)CN1c2ccccc2Sc2ccccc21 ; OCCN1CCN(CCCN2c3ccccc3Sc3ccccc32)CC1]

The compounds disclosed herein can be readily prepared with methods known in the field of organic chemistry. For instance, the synthetic routes reported in publications (e.g. US Patent Application No. 20200009132) can be adopted to the synthesis of the compounds of Formula I. The disclosure of such publication is hereby incorporated by reference.

Diaryl amines with a formula of R—NH—R (two Rs can be linked up in some compounds) such as 3,6-difluoro-9H-carbazole, 3,6-bis(trifluoromethyl)-9H-carbazole, 3,6-dimethyl-9H-carbazole, and tert-butyl piperazine-1-carboxylate can be prepared according to literature procedures (e.g. Tetrahedron 2008, 64, 6038-6050; US 20120022096; J. Org. Chem 2008, 73, 6513-6520; J. Med. Chem, 2003, 46, 4365-4368). As an alternative to Pd-catalyzed aminations, requisite diaryl amines can be conveniently prepared using Knochel's procedure (J. Am. Chem. Soc. 2002, 124, 9390-9391). The entire disclosure of these references are hereby incorporated by reference.

Without being limited by any particular theories, it is postulated that the compounds of Formula I or a pharmaceutical acceptable salt thereof inhibit Bcl-2-associated x-protein (BAX)-mediated cell death, thereby enhanced the viability of cells, tissues, and organs ex vivo.

The composition may be in any suitable form such as a solution, a suspension, and a gel. The composition may also include one or more additional components. Nonlimiting examples include a biological medium, buffer, solvent, co-solvent, vehicle, rheological agent, antioxidant, cell-impermeant constituent, and cell-permeant constituent.

The compound of Formula I can be dissolved in any suitable solvent for the preparation of the composition. Non-limiting examples of the solvent include N-Methyl-2-pyrrolidone (NMP), dimethyl sulfoxide (DMSO), polypropylene glycol, polyethylene glyol, dimethyl isosorbide (DMI), glycerin, and propylene carbonate, and any combination thereof. The composition may also include one or more additional agents, including for example, pentafraction, lactobionic acid, potassium phosphate, magnesium sulfate heptahydrate, raffinose pentahydrate, adenosine, allopurinol, total glutathione, potassium hydroxide, and sodium hydroxide in aqueous or non-aqueous solution or suspension. Various other known solvents and/or agents can also be incorporated in the composition. Further examples of the solvents and agents include those disclosed in U.S. Pat. Nos. 8,617,802, 8,288,084, and US Patent Application No. 20070009880, the entire disclosure of which are hereby incorporated by reference.

The amount or concentration of one or more compounds of Formula I in the composition may vary depending on the specific biological material to be stored, maintained or transported and can be readily adjusted in view of the examples disclosed herein and general knowledge available to public. In some embodiments, the compound is in a solution or suspension at a concentration ranging from about 0.1 μg/mL to about 1000 μg/mL, from about 0.1 μg/mL to about 500 μg/mL, from about 0.1 μg/mL to about 100 μg/mL, from about 0.5 μg/mL to about 50 μg/mL, from about 1 μg/mL to about 30 μg/mL, from about 2 μg/mL to about 20 μg/mL or from about 5 μg/mL to about 15 μg/mL. Nonlimiting examples of the concentration of the compound include about 0.5, about 1, about 2, about 5, about 10, about 15, about 20, about 25, about 30, about 40, about 50 μg/mL, and about 100 μg/mL. In some embodiments, the amount of the compound ranges from about 0.1 to about 100, from about 0.5 to about 50, from about 1 to about 20, or from about 2 to about 10 mg/kg of the weight of the biological material. Nonlimiting examples of the amount of the compound include about 0.1, about 0.5, about 1, about 2, about 5, about 10, about 15, or about 20 mg/kg of the weight of the biological material.

A bio-compatible buffer system or an isotonic buffer system can be included in the composition. For instance, a buffer system that maintains a physiologically acceptable pH, from about pH 6 to about pH 8, under the varying temperature conditions the tissue is subjected to before, during, and after freezing and that is compatible with the other solution constituents and the tissue may be used. Examples of buffers include phosphate buffers, such as phosphate-buffered saline (PBS); organic buffers, such as N-(2-hydroxyethyl)piperazine-N′-(2-ethanesulfonic acid) (HEPES) buffered saline, morpholine propanesulfonic acid (MOPS) buffered saline, and tris(hydroxymethyl) aminomethane (TRIS) buffered saline; and saline buffers such as borate, bicarbonate, carbonate, cacodylate, or citrate ions; or mixtures thereof. In some embodiments, a buffer system uses phosphate-buffered saline to maintain pH from about 7 to about 8, or at about 7.4 at about 25° C.

The composition may also include a cell-impermeant constituent that is compatible with the other constituents of the composition. While not wishing to be bound by any particular theory, the cell-impermeant constituent is believed to reduce damage to the surface of the tissue during freezing and thawing. Preferably, the cell-impermeant constituent has an increased hydrophobic nature in relation to the other solution constituents. While not wishing to be bound by any particular theory, it is believed that the cell-impermeant constituent forms a thin film on the surface of the tissue and may protect the surface of the tissue from abrasion and deformation that could otherwise occur when the crystalline ice matrix forms during freezing. The cell-impermeant constituent may also modify the response of the external membrane of the tissue to changes in osmotic pressure and ionic strength of the cryopreservation solution during freezing and thawing.

While any cell-impermeant constituent that is compatible with the other solution constituents and the tissue to be preserved may be used, preferable cell-impermeant constituents include, but are not limited to, proteins, serums, monosaccharides including sucrose, trehalose, polysaccharides including dextran, agrose, and alginate, long-chain polymers including polyvinylpyrrolidones (PVP), hydroxyethyl starch (HES), derivatives thereof, and mixtures thereof. More preferred cell-impermeant constituents include polyvinylpyrrolidones, hydroxyethyl starches, their derivatives, and mixtures thereof. At present, polyvinylpyrrolidone having a molecular weight of about 17,000 (weight average) is an especially preferred cell-impermeant constituent. Although FBS may be used in addition to or as a replacement for these cell-impermeant constituents, preferable cryoprotectant solutions in accord with the present invention do not include FBS.

A cell-permeant constituent can also be included to serve as an intracellular or tissue-permeating agent. While not wishing to be bound by any particular theory, the cell-permeant constituent is believed to perform at least two desirable functions regarding preservation of the tissue. First, due to the cell-penetrating ability of the solvent, in addition to functioning to protect the exterior surfaces of the tissue during freezing, it can replace water in the interior of the tissue. By replacing at least a portion of the water in the interior of the tissue, it is believed that the formation of the crystalline ice matrix during freezing is interrupted due to interference with the hydrogen-bonding interaction between individual water molecules. Thus, damage to the water-containing interstices of the tissue may be reduced because at least a portion of the water does not crystallize and expand upon freezing. Additionally, by reducing ice formation, it is believed that less water is transported out of the cells during freezing, thus reducing tissue damage from osmotic dehydration. A second desirable feature of the cell-permeant constituent is its ability to lower the freezing point of the composition in relation to water. Preferable cell-permeant constituents are also miscible in water.

Any cell-permeant constituent that is compatible with the other constituents and the tissue to be preserved may be used. Nonlimiting examples include alcohols, such as propanediol, isopropanol, ethanol, t-butanol, mannitol, and glycerol; glycols, such as ethylene glycol and propylene glycol; trimethylamine; acetate; aldoses; ketones; xylose; erythrose; arabinose; ribose; glucose; fructose; galactose; and mixtures thereof.

The composition may further include a radical scavenger to reduce tissue damage from energetic free radicals that form for example during irradiative sterilization of the tissue. While not wishing to be bound by any particular theory, these radical species are believed to form in the fluid boundary layer surrounding the tissue. Not only may free radicals cause crosslinking, oxidation, and other damage during irradiation, but long-lived (persistent) radicals may be formed that cause additional damage during thawing of the tissue. As previously mentioned, when the tissue thaws, the mobility of any persistent radicals present in the frozen matrix can increase, potentially increasing the amount of tissue damage.

Nonlimiting examples of radical scavengers include acids and salts that ionize in water. Preferable radical scavengers include, but are not limited to, sodium ascorbate, carotenoids, l-ascorbic acid, d-isoascorbic acid, sodium sulfite, sodium metabisulfite, sulfur dioxide, nicotinic acid, nicotinic acid amine, cysteine, glutathione, sodium nitrate, sodium nitrite, flavonoids, selenium, alpha-lipoic acids, acetyl cysteine, water-soluble tocopherol derivatives including sodium Vitamin E phosphate (VEP), lauryl imino dipropionic acid tocopheryl phosphate, tocopheryl glucoside, tocopheryl succinate, Tocophersolan (tocopheryl polyethylene glycol 1000 succinate), Tocophereth-5,10,12,18, and 50 (polyethylene glycol (PEG) tocopheryl ethers), Lazaroids, ubiquinone (coenzyme Q10) butylated hydroxytoluene (BHT), butylated hydroxyanisole (BHA), analogs thereof, isomers thereof, derivatives thereof, and mixtures thereof. More preferred radical scavengers include sodium ascorbate, water-soluble derivatives of ascorbate, carotenoids, and mixtures thereof.

Additional nonlimiting examples of antioxidants suitable for the practice of the present invention include one or more compounds selected from the group consisting of 2-tert-butyl-4-hydroxyanisole, 3-tert-butyl-4-hydroxyanisole, or combinations thereof (e.g., butylated hydroxyanisole, BHA); 2,6-di-tert-butyl-4-methylphenol [a.k.a., butylated hydroxytoluene (BHT)], 2,6-di-tert-butyl-p-cresol, (DBPC); ascorbic acid; ascorbate; α-tocopherol; ubiquinol-6; ubichromenol-6; α-tocopherol hydroquinone; α-tocopherol acetate; β-carotene (3,7,12,16-tetramethyl-1,18-bis(2,6,6-trimethyl-1-cyclohexenyl)-octadeca-1,3,5,7,9,11,13,15,17-nonaene); vitamin A (retinol); vitamin B1 (thiamine); vitamin B2 (riboflavin); vitamin B3 (niacin); vitamin B5 (pantothenate); vitamin B6 (pyridoxine/pyridoxal); vitamin B7 (biotin); vitamin B9 (folic acid); vitamin B10 (p-aminobenzoic acid); vitamin B12 (cobalamin/dibencozide); and one or more green tea extracts (including e.g., but not limited to, (−)epigallocatechin-gallate; (−)gallocatechin-gallate; (−)epicatechin-gallate; (−)epigallocatechin; (+)gallocatechin; (−)epicatechin; and (−)catechin).

The composition may also include a biomembrane-sealing agent in an amount effective to extend, lengthen, or prolong the post-harvest and/or pre-implantation viability of an explanted population of mammalian cells, tissue, or a recovered mammalian organ. Nonlimiting examples of these agents include for example, hydrophilic polymers such as poly (oxyethylene) (POE), poly (alkylene glycol) (PAG), poly (ethylene glycol) (PEG), poly (ethylene oxide) (PEO), polyvinyl alcohol (PVA), amphipathic polymers including, but not limited to, pluronics, poloxamers (including poloxamer P-188 [aka CRL-5861] and available from CytRx Corp. (Los Angeles, Calif), as well as methylcellulose, sodium carboxymethyl cellulose, hydroxyethyl starch (HES), polyvinyl pyrrolidine (PVP), and dextrans. Some biomembrane-sealing agents including HES and PEG have shown effective cryopreservative abilities in various organ transplantation studies.

### Methods

An aspect of this patent document provides a method of inhibiting cell death in a biological material ex vivo, for example, during cold storage and/or after a period of warm injury. The method includes contacting the biological sample with a composition comprising an effective amount of a compound of Formula I or any of its sub-formula (e.g. I-a, I-b, I-c, I-d and illustrated compounds) or a pharmaceutically acceptable salt thereof for a certain period of time. The method may also include maintaining the population of mammalian cells or the mammalian tissue or organ in the composition substantially at a temperature, for example, ranging from about −30° C. to about 10° C., from about −20° C. to about 0° C., or from about −20° C. to about 30° C. for a certain period of time. During the period of contacting the sample with the composition or maintaining the sample within the composition, 60% or more of the the biological material remains viable.

The concentration or amount of the compound of Formula I or its salt in the composition is as described above. The sample can be submerged, partically or completedly, in the composition, coated by the composition, or in contact with the composition in any manner suitable to maintain its viability for a desirable period of time.

In some embodiments, prior to the period of contacting the biological sample or maintaining it with the composition, there includes optionally a step of flushing, rinsing or treating it with the composition at the time of sample harvest. The amount of the compound or its salt for this optional step can be determined by the weight or nature of the sample. For instance, the amount of the compound can range from about 0.1 to about 100, from about 0.5 to about 50, from about 1 to about 20, or from about 2 to about 10 mg/kg of the weight of the biological material. Nonlimiting examples of the amount of the compound include about 0.1, about 0.5, about 1, about 2, about 5, about 10, about 15, or about 20 mg/kg of the weight of the biological material. This flushing or treatment step may be repeated by one, two or more times.

Using the compositions disclosed herein, the present methods now permit maintenance of biologic activity and/or cellular viability of explanted tissues even when stored under appropriate conditions for periods of time of from several days, to several weeks, and even up to and including several months post-harvest. In some embodiments, the amount of the composition or the compound of Formula I or a pharmaceutically acceptable salt thereof is selected so that the cell death is reduced by more than 10%, more than 20%, more than 30%, more than 40%, more than 50% or more than more than 60% in comparison with a reference without the composition over a period of about 6 hours, about 12 hours, about 24 hours, about 36 hours, about 48 hours, about 60 hours, or about 72 hours, or for a period of 1, 2, 5, 7, 10, 12, 14, 15, 30, 60, 90, 120, 150, or more days at a temperature of for example about −30° C., about −20° C., about −10° C., about −5° C., about 0° C., about 5° C., about 10° C., about 15° C., about 20° C. or about 25° C.

This method can be applied to storing and/or transporting one or more populations of mammalian cells, tissues, or organs, under conditions that permit long-term retention of viability and/or biological activity and/or function. The method is capable of keeping the population of mammalian cells or the mammalian tissue or organ viable both during storage and immediately thereafter maintaining such cells or tissues/organs in a physiologic state that is suitable for their implantation into a selected recipient mammal.

In some embodiments, the present methods permit retention of 60% or more (e.g. 65%, 70%, 75%, 80%, 85%, 90%, 95% or more) of the initial post-harvest viability of a biological material when stored in such compositions and maintained under appropriate environmental conditions over a period of about 6 hours, about 12 hours, about 24 hours, about 36 hours, about 48 hours, about 60 hours, or about 72 hours, or for a period of 1, 2, 5, 7, 10, 12, 14, 15, 30, 60, 90, 120, 150, or more days at a temperature of, for example, about −30° C., about −20° C., about −10° C., about −5° C., about 0° C., about 5° C., about 10° C., about 15° C., about 20° C. or about 25° C. In illustrative embodiments, various biological materials may be prepared, stored, and transported under conditions that permit the recovered cells, tissues, or organs to retain significant viability (e.g., 75%, 80%, 85%, 90%, or 95% of their initial post-harvest viability) when compared to storage of similar biological samples in conventional buffers, organ transport solutions, or mammalian growth media alone.

In some embodiments, more than about 60%, more than about 65%, more than about 70%, more than about 75%, more than about 80%, more than about 85%, more than about 90%, more than about 95% or more than about 98% of the biological material (e.g. a population of mammalian cells, tissue, organ, etc.) remains viable after maintaining the material in the composition over a period of about 6 hours, about 12 hours, about 24 hours, about 36 hours, about 48 hours, about 60 hours, or about 72 hours, or for a period of 1, 2, 5, 7, 10, 15, 30, 60, 90, 120, 150, or more days at a temperature of, for example, about −30° C., about −20° C., about −10° C., about −5° C., about 0° C., about 5° C., about 10° C., about 15° C., about 20° C. or about 25° C. In some embodiments, the biological remains substantially viable after maintaining the material in the composition over a period of about 6 hours, about 12 hours, about 24 hours, about 36 hours, about 48 hours, about 60 hours, about 72 hours, or longer than 80 hours. In those conditions, a biological material that is “substantially viable” includes, but is not limited to the organ, tissue or population of cells, that when stored, maintained, and/or transported in one or more of the compositions disclosed herein, that are at least about 95% viable, at least about 96% viable, at least about 97% viable, at least about 98% viable, or even at least about 99% viable.

It is also contemplated that the disclosed compositions will find particular utility in methods for preserving sufficient biological functional and retaining sufficient cellular viability and/or tissue integrity in poorly-perfusable mammalian tissues that have previously not been amenable to long-term storage. Through use of the present compositions, tissues that were previously only biologically-viable for implantation following short-term (e.g., several hours to several days) storage, may now be prepared that are substantially biologically active and amenable to intermediate-term (e.g., several days to several weeks) and even extended or long-term (e.g., several weeks to several months or more) storage. Such methods thereby significantly extend the conventional harvest-to-implantation “window of opportunity,” and provide novel methods for extending the usable “shelf-life” of recovered tissues or cultured cell populations from several hours to many weeks to even several months or longer.

While the methods and compositions of the present invention are contemplated to be useful in the storage and viability-preserving function of a variety of animal cells, tissues, and organs outside the living body of the donor, such methods and compositions are particularly suited for the harvest, storage and transport of mammalian cells, tissues, and organs. Especially relevant are those cells, tissues, or organs that are recovered from suitable living or cadaveric donor mammals and are destined for implantation into suitable mammalian recipients.

Exemplary types of mammalian cells which may be recovered, stored, and/or transported using one or more of the methods and compositions described herein include, but are not limited to: chondral cells, cartilagenous cells, osteochondral cells, islet cells, osteogenic cells, neural cells, bone cells, bone marrow cells, adipose cells, fibroblasts, muscle cells, blood, blood components, stem cells, and embryonic stem cells.

Exemplary types of mammalian tissues which may be recovered, stored, and/or transported according to the present invention include, but are not limited to, skin, cartilage, tendons, ligaments; fascia, tibialis, patellas and other bones, heart valves, semi-tendinous tissues, blood vessels, vertebral discs, corneas, lenses, meniscus, hair, adipose tissue, fibrous tissue, neural tissue, connective tissue, and striated, smooth, or cardiac muscle tissue. The cells or tissues may be recovered from human or animal subjects and are then processed and/or cryopreserved (frozen) for later implantation. Allograft tissues, including, but not limited to, heart valves and portions of heart valves, aortic roots, aortic walls, connective tissues including fascia and dura, vascular grafts (including arterial, venous, and biological tubes), and orthopedic soft tissues, such as boned- or non-boned tendons or ligaments, are often subjected to cryogenic preservation. In this manner, a ready supply of these valuable tissues can be made available for later implantation into mammals, especially humans. In addition, viable xenograft tissues from transgenic animals or tissues developed from human or non-human cells that may include differentiated cell types, stem cells, or genetically-modified cells of various origins may be appropriately processed, cryopreserved, and stored for later implantation. Additional examples include engineered cells of tissues or tissue engineered constructs.

Explanted animal tissues, cell populations, and recovered mammalian organs stored or maintained by any one of the methods or processes disclosed herein, or any explanted mammalian cells, tissue, or organ stored in one or more of the disclosed compositions are preferably suitable for implantation into a selected recipient animal, and particularly into a selected recipient mammal. Examples of mammalian species into which the explanted tissue may be transplanted, include, but are not limited to, humans, cattle, horses, sheep, pigs, goats, rabbits, dogs, cats, and non-human primates.

In some embodiment of any method disclosed herein, the cell types may include chondral, cartilagenous, osteochondral, islet, osteogenic, neural, bone, bone marrow, adipose, fibroblast, muscle, blood, and stem cells; the animal tissues may include skin, bone, cartilage, tendon, ligament, vertebral disc, cornea, lens, meniscus, hair, striated muscle, smooth muscle, cardiac muscle, adipose tissue, fibrous tissue, neural tissue, and connective tissue; or the mammalian organs may include cochlea, testis, ovary, stomach, lung, heart, liver, pancreas, kidney, intestine, and eye.

Cell populations, tissues and organs prepared by the processes provided herein may be of any origin, although those of animal origin and of mammalian origin in particular, are preferable. Exemplary explanted biological materials may be obtained from one or more animals, including, but not limited to, bovines, canines, caprines, equines, felines, gallines, humans, lapines, leporines, lupines, murines, ovines, porcines, vulpines, or non-human primates.

The compositions disclosed herein may also be used to perfuse the tissues, organs, or circulatory system of the donor animal prior to harvest (either while the animal is still alive, or alternatively, postmortem). The disclosed compositions may also be used as a wash solution to cleanse the freshly-recovered tissues from the host animal prior to long-term storage, transport, or transplantation.

In the practice of the present invention, it is often desirable to maintain the cells, tissues, or organs in the composition essentially from a time immediately post-harvest until the explant material is readied for transplantation into a recipient mammal. During the interval between harvest and implantation, it is also desirable to monitor and control the environmental conditions, and storage parameters to maintain the integrity, viability, and biochemical activity of the recovered biological material.

In some embodiments, the compositions or methods disclosed herein find particular use in the storage and/or transport of tissues at an ambient storage temperature in the range from about −55° C. to about 25° C., from about −45° C. to about 35° C., from about −25° C. to about 25° C., or from about −15° C. to about 15° C. Preferably, at least a portion of the storage solution remains substantially in an unfrozen, or liquid state. While it is contemplated that slight variation in temperature during the storage/transport process will not adversely affect the integrity, biological function, or cellular viability of the stored tissue or organ, in some embodiments the material is maintained and transported under environmental conditions of approximately −10° C. and about 25° C., or from between about −5° C. and about 20° C., from between about 0° C. and about 15° C., or from between about 0° C. and about 10° C. Nonlimiting examples of the temperature within the aforementioned ranges include about −30° C., about −20° C., about −10° C., about −5° C., about 0° C., about 5° C., about 10° C., and about 15° C.

To preserve the integrity and viability of the biological material to the best extent possible, it may be desirable to contact freshly-recovered cells, tissues, or organs with the disclosed tissue viability-preserving compositions/formulations substantially immediately upon harvest, and to maintain the recovered cells, tissues, or organs in these formulations substantially until immediately prior to implantation. Pre-cooling of the composition to the desired storage temperature prior to contacting it with the recovered cells or tissues will often be desirable.

The method disclosed herein enhances viability of the biological materials during cold storage either with or without a preceding period of warm ischemic injury. A preceding period of warm ischemic injury refers to a period of reduced or no blood flow within the organ or tissue between when life support is withdrawn from the donor and death is declared. The period may range from about 1 minute to about 24 hours, from about 1 hour to about 15 hours, from about 2 hours to about 10 hours, from about 1 minute to about 10 hours, from about 1 minute to about 3 hours, from about 1 minute to about 1 hour, from about 5 minute to about 40 minutes, from about 5 minute to about 20 minutes, or from about 10 minute to about 30 minutes.

In certain circumstances, it may also be desirable to irrigate, infuse, perfuse, or wash the recovered biological material with one or more portions of the compositions immediately upon removal from the living or cadaveric donor organism, and then to subsequently transfer the washed biological material to a fresh aliquot of the composition just prior to storage.

In some circumstances, depending upon the tissue type, and the length of storage, it may also be desirable to periodically decant the “spent” medium of the composition from the stored tissue, and to replenish the storage means with fresh medium. Likewise, it may also be desirable to perform one or more additional perfusion or wash steps after removing the tissue from storage, and immediately prior to implantation into the recipient animal.

In some embodiments of any method disclosed herein, there may also include a step of cryogenically-preserving (i.e., freezing) a population of cells, tissues, or organs using tissue preservative buffers, solutions, or supplemented growth media. The step of freezing the tissue or biological material may optionally include the addition of one or more cryoprotectants or cryopreservative compounds to further permit freezing of the sample, and/or maintenance of the sample at temperatures generally below 0° C. Exemplary cryoprotectants and/or cryopreservative compounds, as used in the context of the present invention may include, but are not limited to, ice-suppressing cryoprotectants (e.g., non-colligative agents such as Supercool X-1000™ and Supercool Z-1000™, 21st Century Medicine, Rancho Cucamonga, Calif.) glycerol, dimethylsulfoxide (DMSO), ethylene glycol, propylene glycol, polyethylene oxide (PEO), acetamide, ethanol, methanol, butanediol, carbohydrates (including sugars such as glucose, fructose, dextrans, sucrose, lactose, and trehalose), polyvinyl alcohols, hydroxyethyl starch, serum albumin, and such like.

In some embodiments, it may also be desirable to provide one or more optional additional steps in method, including, for example, steps that involve freezing and/or thawing of a tissue sample or cell population. Such freezing and thawing steps may be achieved by any conventional manner known to those in the art, (e.g., slowly bringing the temperature of a refrigerated tissue or cell sample down to a suitable sub-zero temperature, or alternatively, slowly bringing the temperature of a sub-zero stored sample up to refrigerated (and, optionally, to either room or recipient body temperature immediately prior to implantation). Such additional steps in the method may employ submersion vessels or frozen storage means to prepare the frozen tissue or cell sample, while conventional means such as a heated water bath or such like device, submerging the frozen sample directly into a sample of growth medium, biological buffer, or tissue/organ storage solution (e.g., pre-warmed to the desired temperature), may be employed to bring the temperature of a frozen tissue sample to the desired temperature required for transplanting the biological material into the body of a suitable recipient animal. Additional examples on freezing and thawing are available in U.S. Pat. No. 7,129,035, the entire disclosure of which is hereby incorporated by reference.

Also provided herein is a population of cells, an explanted biological tissue, or a recovered organ that is treated or stored by any one of the methods and processes described herein. Although there is no inherent limitations to the cell, tissue, or organ types that may benefit from being maintained and/or transported in one or more of the disclosed storage compositions, the population of donor cells, tissue(s), or organ(s) are generally of animal origin, and in particular, of mammalian origin. Exemplary donor cell, tissues, and organs include, but are not limited to, those of human, bovine, ovine, porcine, equine, canine, feline, caprine, luprine, or non-human primate origin. In some embodiments, the human may be a patient under the care of a physician or other medical professional, and is, will, or may have been in need of transplantation or one or more cells, tissues, or organs recovered from a suitable donor mammal.

In any embodiment disclosed herein, the viability of cells, tissues, and organs, and particularly those obtained from animals, can be determined by assays that are known to those of skill in the relevant art. For example, the viability of cells is readily determined by using a microscopic assay that is commonly referred to in the art as a “live/dead assay”. In one such assay, the biologic dyes 5-chloromethylfluorescein diacetate and propidium iodide (which differentially stain living and non-living cells) are employed and evaluated by a microscopy-based assay. Such dyes are typically fluorescent, and the fluorescence may be detected and used to produce dual-parameter fluorescence histograms, most typically using fluoromicroscopic techniques to distinguish the living vs. the non-living cells, in which the living and non-living cells each fluoresce at distinctly-different wavelengths.

In some embodiments, to determine the % viability of tissues that have been stored as a function of time, a biological sample may be initially assayed for viability (typically within 10 minutes, within 30 minutes, within 1 hour, within 2 hours, within 4 hours, within 8 hours, within 12 hours, within 24 hours, within 48, or within 72 hours of harvest from the donor animal) to determine an “initial viability.” Subsequent viability determinations are then made on the tissue over a period of time to determine “current viability.” The % viability can therefore be determined at any time post-harvest using the following equation:

[(current viability)/(initial viability)]×100=Percent viability

If desired, multiple samples may be analyzed and averaged both at initial assay, and/or during subsequent analyses to determine an “average viability” of the recovered tissue. Alternatively, the determination of tissue, cell, or organ viability may also include one or more biochemical or anatomical assays that are known in the art, and which provide qualitative and/or quantitation evidence of the biological activity or functionality of the explanted tissue once it is introduced into the recipient animal. Additional assays and detection methods are provided in U.S. Pat. No. 9,737,071, the entire disclosure of which is hereby incorporated by reference.

The compositions and methods disclosed herein can also be applied to inhibiting cell death in a biological material (e.g. a population of mammalian cells, tissue, organ, etc) ex vivo or prolonging the viability of the biological material ex vivo. The method can be practed under the same conditions (including temperature range, amount and concentration of the compound of Formula I, and other factors) and therefore achieving the same level of viability for the biological material as descried above.

### Kit

Another aspect of the patent document provides a kit including the composition descried herein and a manual or instruction on the use of the kit for storing a biological material. The manual includes for example protocols detailing the recommended use of the disclosed compositions as a rinse solution prior to, during, or subsequent to the harvesting, storage, handling, and/or transportation of the biological material using one or more of the disclosed compositions.

The kit may also optionally include one or more containers for storing the biological sample, or one or more devices for obtaining, explanting, or implanting the biological material into a suitable recipient. Such kits may also be prepared for convenient commercial packaging, sale, use, and transport. Exemplary packaging means for harvest, storage, and/or delivery of the biological material include, but are not limited to, gas-permeable or gas-impermeable containers, with or without a gaseous headspace. Such packaging means may incorporate the use of clear or opaque plastics, as well as hard, or flexible packaging. The standard methods for collecting, preparing, storing, and transporting explanted biological materials are considered to be within the purview of the artisan skilled in the transplantation arts, and as such, are not described in further detail herein.

For example, such a kit may comprise, in suitable container means, a stock solution of comprising a tissue viability-prolonging composition disclosed herein, optionally in combination with a commercially available buffer, growth medium, diluent, or storage solution. Such kits may comprise any convenient amount of such components, and the components may even be pre-measured such that the entire contents of the stock solution is added to the entire amount of storage medium solution in the kit to produce a one-step, or “ready-to use” final working solution obtained simply by combining the two pre-measured amounts.

To facilitate timely and accurate combination of the ingredients, the kit may also provide means for combining two or more ingredients, such as for example, a sterile syringe or other suitable sterile delivery means for adding a smaller quantity of one solution into a larger quantity of a second solution (e.g., a concentrated form of the supplement may be prepared in a sterile delivery means, which is then used to introduce a quantity of the supplement to a separate container of standard commercially-available growth medium or buffer solution to form the final working compositions as disclosed herein.

In certain embodiments, the compositions and the kit disclosed herein, as well as one or more biological samples stored therein, may also be useful in the prophylaxis, therapy, or amelioration of symptoms of one or more diseases, dysfunctions, defects, injuries, or disorders in a mammal. Such compositions may also find particular use in the preparation of a medicament for prophylactic, therapeutic, and/or ameliorative regimens, particularly in the harvesting of biological materials from donor animals, or in the surgical transplantation of such materials into selected recipients.

### EXAMPLES

### Example 1

Compound synthesis. The scheme below illustrates a synthesis route to one of the compounds disclosed herein. Multiple alternative routes are certainly available in view of well-known synthetic methodologies reported in literature.

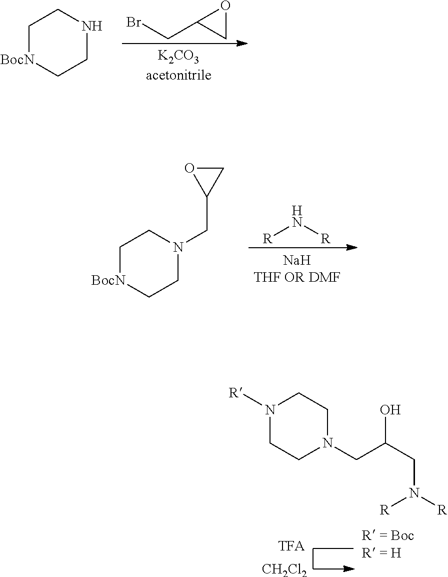

In this scheme, tert-Butyl piperazine-1-carboxylate was added to 2-(bromomethyl)oxirane to give intermediate. Amines were deprotonated using sodium hydride and used to open the epoxide. The resulting secondary alcohol was subjected to standard Boc deprotection conditions to obtain the target compounds. (THF=tetrahydrofurane; DMF=N,N-dimethylformamide, TFA=trifluoroacetic acid; Boc=tert-butylcarboxylate)

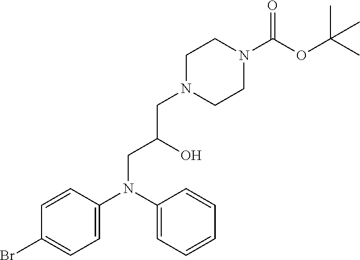 [SMILES: CC(C)(C)OC(=O)N1CCN(CC(O)CN(c2ccccc2)c2ccc(Br)cc2)CC1]

Synthesis of tert-butyl 4-(3-((4-bromophenyl)(phenyl)amino)-2-hydroxypropyl)piperazine-1-carboxylate. In a flame-dried 60 mL Centrifuge tube with septum and stir bar, sodium hydride (60% dispersion in mineral oil) (258 mg, 6.45 mmol, 1.60 equiv) was suspended in dry DMF (5.7 mL) under an argon atmosphere. In a separate dry and argon-flushed tube, 4-bromo-N-phenylaniline (1.50 g, 6.05 mmol, 1.50 equiv) was dissolved in dry DMF (18.4 mL). The NaH-suspension was cooled to 0° C. (ice bath) and the diphenylamine solution was slowly added over ca 10-15 min. A color change to bright yellow, later green was observed. After 20 min, the mixture was warmed to room temperature (RT) and stirred for an additional 30 min. The mixture then was cooled to 0° C. again.

A solution of tert-butyl 4-(oxiran-2-ylmethyl)piperazine-1-carboxylate (0.977 g, 4.03 mmol) in dry DMF (2.83 mL) was added over 5 min. The mixture was stirred at 0° C. for 10 min, then warmed to RT and stirred at this temperature. Thin layer chromatography (TLC; 1:1 hex:EtOAc) was used to monitor the reaction progress. After TLC indicated full conversion, the mixture was poured onto satd. aq. sodium bicarbonate (75.0 mL), extracted with EtOAc (150 mL and 2×75.0 mL). Combined organic layers were dried (MgSO₄), filtered and evaporated in vacuo. The crude residue was purified on an Isco CombiFlash (silica gel, EtOAc in hexanes, 30%→60%) (605 mg, 1.23 mmol, 31%) was obtained as off-white solid. The corresponding O-acetate (1.01 g) was isolated as a side-product. The acetate had presumably formed on the loading column from the desired product and ethyl acetate, triggered by heat formed when DMF remainders in the crude material came in contact with the silica. This behavior was not observed on a significant level in smaller scale reactions and can be avoided by more thorough drying of the crude in high vacuum (10′ mbar). The acetate can be conveniently hydrolyzed by treatment with potassium carbonate (2.0 equiv) in methanol (0.11 M) for 2 h to give another crop of the desired product (685 mg, 35%).

TLC: R_{f} 0.37 (1:1, hex:EtOAc). ¹H-NMR (600 MHz, CDCl₃): δ 7.32-7.28 (m, 4H), 7.08 (d, J=7.9 Hz, 2H), 7.03 (t, J=7.4 Hz, 1H), 6.88 (d, J=8.9 Hz, 2H), 4.00 (dd, J=10.3, 5.7 Hz, 1H), 3.79-3.71 (m, 2H), 3.44-3.38 (m, 4H), 3.31 (s, 1H), 2.53-2.52 (m, 2H), 2.41 (dd, J=12.4, 3.5 Hz, 1H), 2.34 (m, 3H), 1.45 (s, 9H). ¹³C-NMR (151 MHz, CDCl₃): δ 154.8, 147.9, 147.8, 132.2, 129.7, 122.9, 122.7, 121.6, 113.2, 80.0, 65.1, 62.27, 56.8, 53.2, 43.8 (d, br), 28.6. ESI-MS m/z (rel int): (pos) 514.1 ([M(⁸¹Br)+Na]⁺, 18), 512.1 ([M(⁷⁹Br)+Na]⁺, 14), 492.1 ([M(⁸¹Br)+H]⁺, 100), 490.1 ([M(⁷⁹Br)+H]⁺, 95), 436.0 (14), 435.0 (18).

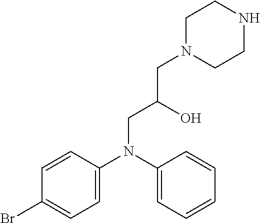 [SMILES: OC(CN1CCNCC1)CN(c1ccccc1)c1ccc(Br)cc1]

Synthesis of 1-((4-bromophenyl)(phenyl)amino)-3-(piperazin-1-yl)propan-2-ol. tert-Butyl 4-(3-((4-bromophenyl)(phenyl)amino)-2-hydroxypropyl)piperazine-1-carboxylate (605 mg, 1.23 mmol) was dissolved in dichloromethane (20.6 mL). The flask was purged with argon for a few minutes, and TFA (5.23 mL, 67.8 mmol, 55.0 equiv) was added at RT and the mixture stirred at the same temperature. TLC analysis of a reaction aliquot (micro-workup, satd. aq. NaHCO₃/EtOAc) indicated complete conversion after 1 h 05′. The mixture was poured on sodium bicarbonate (6.22 g, 74.0 mmol, 60.0 equiv) in 20.0 mL water and stirred vigorously at RT for 40 min. More sodium bicarbonate was added as needed to bring the aqueous layer to pH=8. The layers were separated and the aqueous layer was extracted with EtOAc (2×75.0 mL). The combined organic layers were washed with satd. aq. NaHCO₃ (50.0 mL) and brine (50.0 mL), dried (MgSO4), filtered and evaporated in vacuo. The residue was taken up in CH₂Cl₂, filtered through syringe filter (pore size), then evaporated in vacuo and dried in high vacuum. Ethyl-3-oxo-3-phenyl-2-(2-(thiazol-2-yl)hydrazono)propanoate (470 mg, 1.20 mmol, 98%) was obtained as a sticky, light brown solid.

TLC: R_{f} 0.09 (95:5, CH₂Cl₂:MeOH). ¹H-NMR (600 MHz, CDCl₃): δ 7.33-7.28 (m, 4H), 7.09 (dd, J=8.6, 1.1 Hz, 2H), 7.03 (tt, J=7.3, 1.1 Hz, 1H), 6.89 (d, J=9.0 Hz, 2H), 4.03-3.98 (m, 1H), 3.78-3.70 (m, J=5.7 Hz, 2H), 2.94-2.87 (m, 4H), 2.63-2.60 (m, 2H), 2.44-2.38 (m, 3H), 2.30 (dd, J=12.4, 10.1 Hz, 1H). ¹³C-NMR (151 MHz, CDCl₃): δ 147.9, 147.8, 132.2, 129.7, 122.9, 122.7, 121.6, 113.2, 64.8, 62.7, 56.9, 54.2, 46.0. ESI-MS m/z (rel int): (pos) 392.09 ([M(⁸¹Br)+H]⁺, 100), 389.9 ([M(⁷⁹Br)+H]⁺, 99).

### Example 2

Methods: TUNEL Staining and Quantification. Frozen kidney sections were fixed with 4% paraformaldehyde and stained according to standard protocols for the In Situ Cell Death Detection Kit, TMR red (Millipore Sigma). The number of DAPI positive and TUNEL positive cells were quantified using cell counter macros in ImageJ across mouse, pig, and human samples.

Murine and Porcine Kidney Recoveries. Mouse and pig kidneys were recovered using standard organ recovery protocols. Prior to flushing the abdominal cavity with preservation solution, mice and pigs were given heparin to help increase flow and prevent clotting within the system.

Bax KO or Wildtype mice (3× per group) were euthanized and then organs recovered following standard organ transplant procedures. Mouse kidneys were preserved in University of Wisconsin's Preservation fluid (pentafraction [50 g/L], lactobionic acid [35.83 g/L], potassium phosphate [3.4 g/L], magnesium sulfate heptahydrate [1.23 g/L], raffinose pentahydrate [17.83 g/L], adenosine [1.34 g/L], allopurinol [0.136 g/L], total glutathione [0.922 g/L], potassium hydroxide [5.61 g/L], sodium hydroxide, 5N [5.0 mL/L], water [q.s.]) for 0, 24, 48 and 72 hrs. At the proscribed time points, kidneys were snap frozen for subsequent cell death assessment by fluorescent TUNEL staining of frozen sections collected on an EVOS2 scope. Quantification of TUNEL staining was performed with custom matlab code following standard image analysis procedures. This experiment of TUNEL staining for cell death in cold stored mouse kidneys showed that genetic deletion of BAX is markedly cytoprotective.

### Example 3

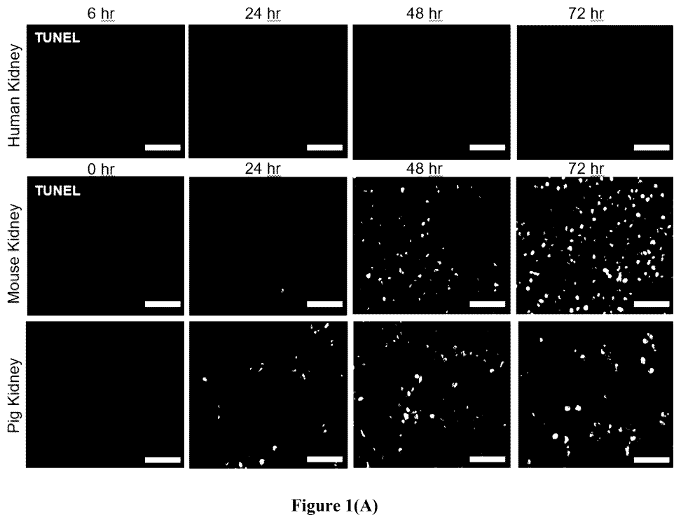

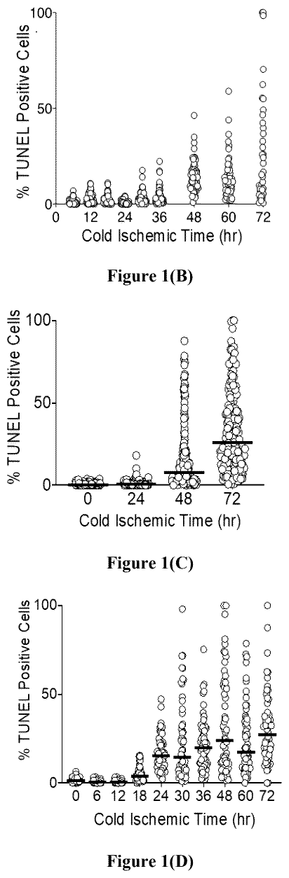

To assess the extent of cell death during cold preservation we repeatedly biopsied a series of eleven human kidneys at times from 12 to 72 hours of cold storage. Kidneys from wild type adult mice and pigs exhibit the same cell death phenotype as assessed by TUNEL staining (FIGS. 1(A)-1(D)).

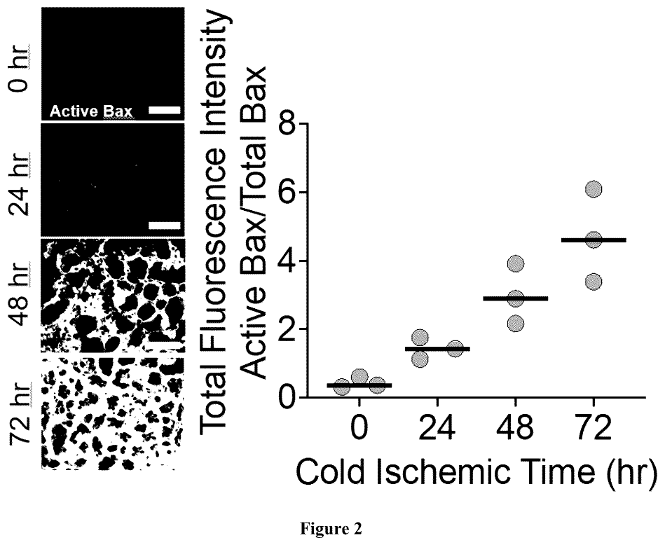

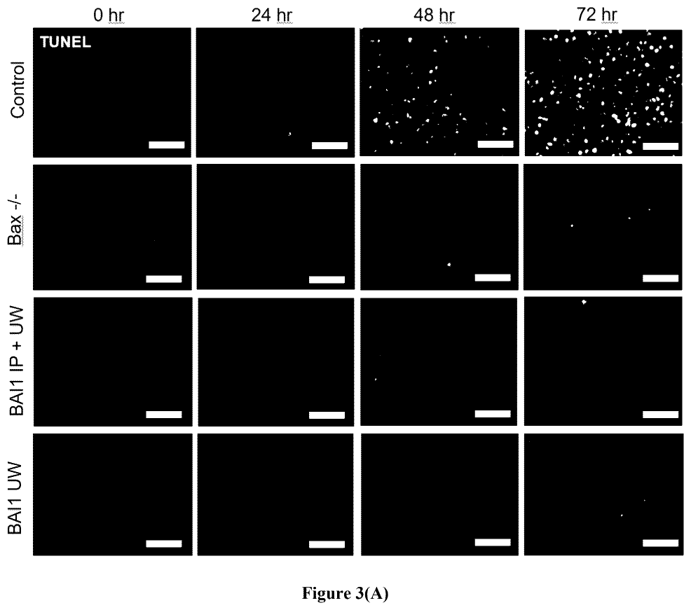

Cell death arises in mouse concurrent with Bax activation during prolonged cold storage (FIG. 2). In mouse kidneys the appearance of cell death with TUNEL staining is concurrent with increased activation of Bax over time as total amounts of Bax remained stable. This result was visualized using immunofluorescent co-staining of Bax Antibody 2772 (Cell Signaling) and Bax Antibody Clone 6A7 (BD Biosciences) in mouse kidneys over 72 hours of cold storage. In mouse kidneys the appearance of cell death with TUNEL staining was concurrent with increased activation of Bax over time as total amounts of Bax remained stable. This result was visualized using immunofluorescent co-staining of Bax Antibody 2772 (Cell Signaling) and Bax Antibody Clone 6A7 (BD Biosciences) in mouse kidneys over 72 hours of cold storage.

### Example 4

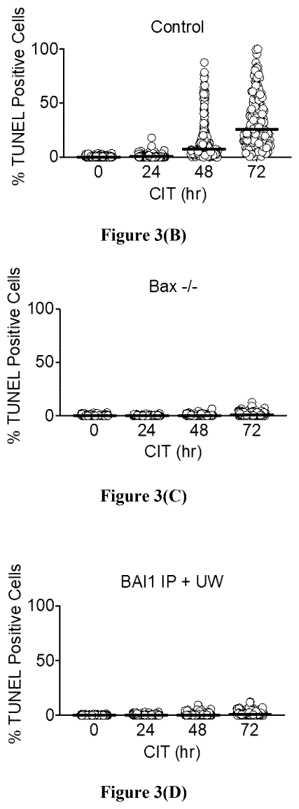

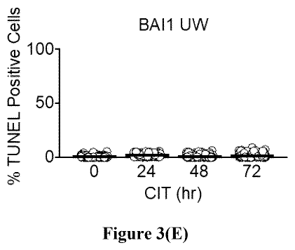

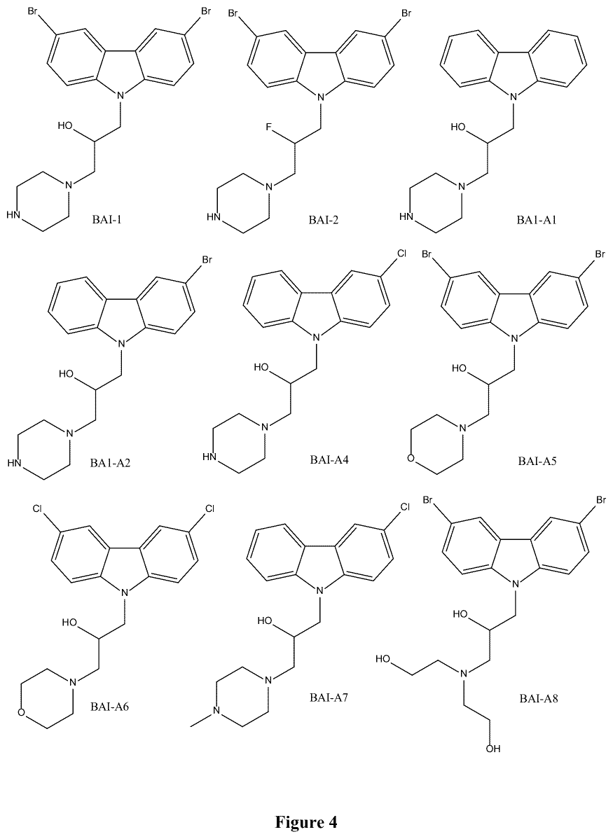

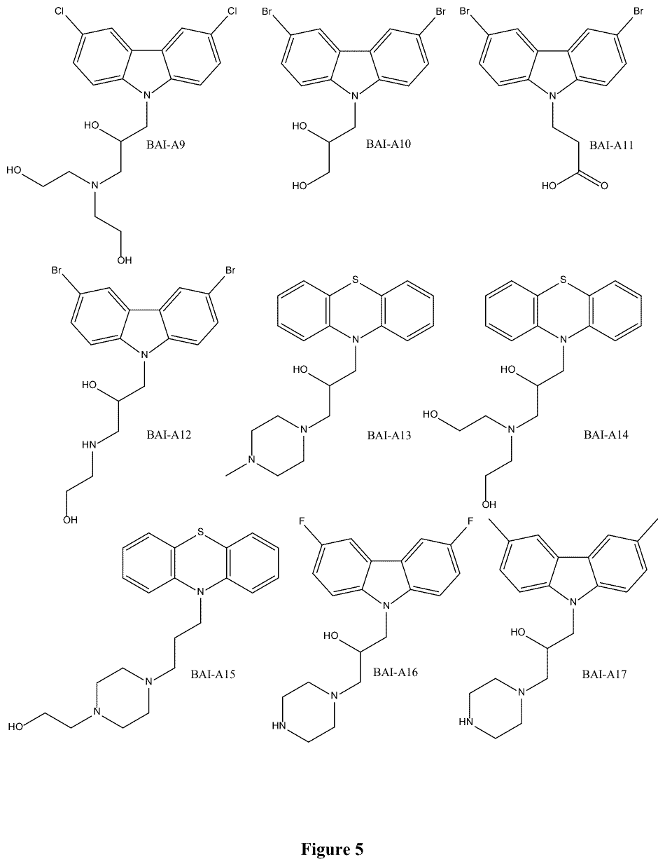

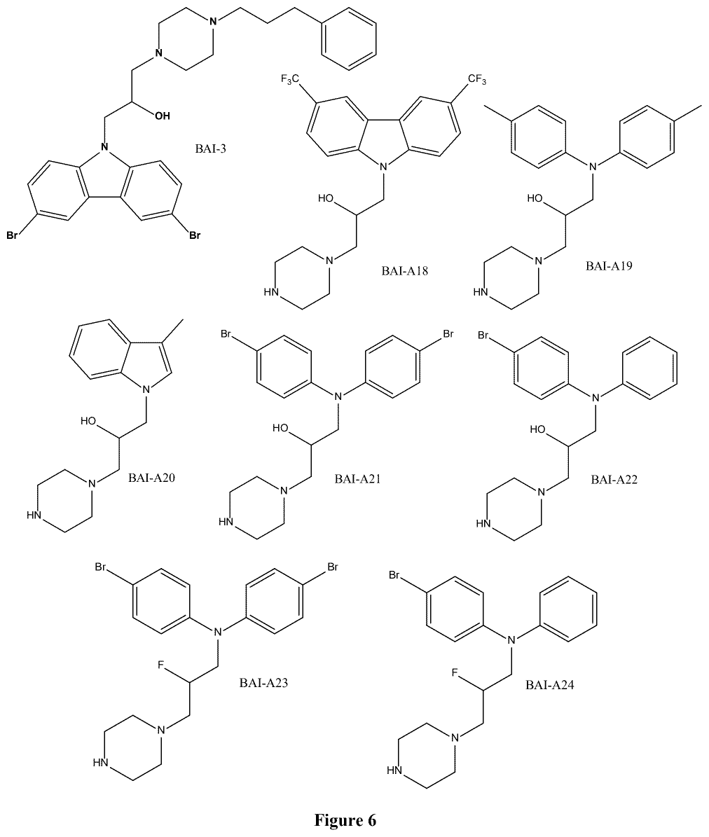

Bax inhibition exclusively abrogates cell death during prolonged cold storage in mice and pigs (FIGS. 3(A)-3(E)). It was observed that both Bax inhibition either using Bax knockout mice or BAI1 supplemented cold preservation solution at time of organ recovery prevents cell death during prolonged cold storage (72 hours) in mice and pigs. Mice were flushed with BAI supplemented University of Wisconsin solution (2 mg/kg total mouse weight) at time of organ recovery. Mouse kidneys were stored in the same concentration of BAI1 supplemented preservation solution that was used to flush the mice at time of organ harvest. Pig kidneys were flushed with 500 mL of University of Wisconsin solution supplemented with BAI1 at 10 μg/mL. They were then stored at 4° C. in 500 mL of University of Wisconsin solution supplemented with BAI1 at 10 μg/mL for 72 hours. Other cell death inhibitors (e.g. pan-caspase inhibitor Z-VAD-FMK, ferrostatin-1, necrostatin-1, and cyclosporin-A) delivered at 2 mg/kg total mouse weight in cold preservation solution did not appear to reduce the amount of cell death seen throughout cold storage, and in some instances increased cell death as compared to controls. It was observed that while Bak knockout mice didn't completely abrogate cell death like Bax knockout mice did, cell death was significantly reduced in Bak knockout mice over 72 hours of cold storage as compared to controls.

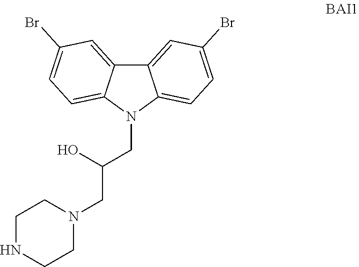 [SMILES: OC(CN1CCNCC1)Cn1c2ccc(Br)cc2c2cc(Br)ccc21]

### Example 5

To better appreciate how kidneys respond to cold ischemia, a series of nine kidneys was biopsied at times from 12 to 72 hours of cold storage. To decreased observed cell death, a Bax inhibitor (BAI1) was tested in a human organ culture model.

To assess the extent of cell death during cold preservation, a series of eleven human kidneys were repeatedly biopsied at times from 12 to 72 hours of cold storage. It was found that kidneys from older, more marginal donors (5/11) had significantly more cell death visualized via TUNEL staining than younger donors (6/11). Cell death increased over time and became apparent in marginal donors between 30-36 hours of cold storage. Human kidneys with the cell death phenotype were also shown to have distinct upregulation of metabolites (e.g. xylose, isothreonic acid, 3-hydroxy-3-methylglutaric acid, conduritol-beta-epoxide, hippuric acid, pseudo uridine, hydroxyproline dipeptide, 4-hydroxyhippuric acid) as compared to the younger, healthier cohort of organs. Preliminary data indicated that tubular epithelial cells are the predominate cell type positive for TUNEL staining as cold time increases.

Additionally, it was found that the tested BAX inhibitor significantly decreased cell death in human organ culture, inhibiting cell death both during cold storage and after a period of warm injury. These data indicate that cell death throughout the course of cold storage can be reduced by pharmacological intervention in marginal organs and thus may improve clinical outcomes.

It will be appreciated by persons skilled in the art that compositions, methods and kits described herein are not limited to what has been particularly shown and described. Rather, the scope of compositions, methods and kits are defined by the claims which follow. It should further be understood that the above description is only representative of illustrative examples of embodiments. The description has not attempted to exhaustively enumerate all possible variations. The alternate embodiments may not have been presented for a specific step or component of the method, and may result from a different combination of described steps or components, or that other alternate embodiments may be available for a step or component, is not to be considered a disclaimer of those alternate embodiments. It will be appreciated that many of those embodiments are within the literal scope of the following claims, and others are equivalent.

1. A method of storing a biological material ex vivo or prolonging the viability of the biological material ex vivo for a period of time, comprising:
contacting the biological sample during the period with a composition comprising an effective amount of a compound of Formula I or a pharmaceutically acceptable salt thereof, 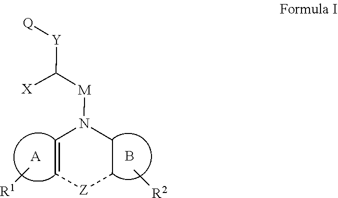
wherein
A is phenyl or a 6-membered heteroaromatic ring having 1, 2 or 3 atoms in the heteroaromatic ring;
B is phenyl, a 6-membered heteroaromatic ring having 1, 2 or 3 heteroatoms in the heteroaromatic ring, or a 6-membered aliphatic ring wherein optionally 1, 2 or 3 ring carbons are replaced with heteroatoms;
Z is void, a bond, O, S, NR^{a}, C(R^{a})₂, S(O)₂ or C(O);
the dashed line between A and B indicates an optional bond;
R¹ and R² are independently selected from the group consisting of H, C₁₋₆ alkyl, F, Cl, Br, I, CN, NO₂, N(R^{a})₂, OC₁₋₆ alkyl, CF₃, COOH, COOC₁₋₆ alkyl, OC(C═O)C₁₋₆ alkyl, OC₁₋₆ alkyl, (C═O) C₁₋₆ alkyl, C₁₋₆ alkylene-OH, SC₁₋₆ alkyl, SOC₁₋₆ alkyl, and SO₂C₁₋₆ alkyl;
X is H, F, Cl, Br, I, CN, SH, NO₂, N(R^{a})₂, OR^{a}, CF₃, COOH, C₁₋₆ alkyl, COO C₁₋₆ alkyl, OCC₁₋₆ alkyl, OCO C₁₋₆ alkyl, O C₁₋₆ alkyl, S C₁₋₆ alkyl, SO C₁₋₆ alkyl, and SO₂ C₁₋₆ alkyl;
X is H, F, Cl, Br, I, CN, SH, NO₂, N(R^{a})₂, OR^{a}, CF₃, COOH, C₁₋₆ alkyl, COO C₁₋₆ alkyl, OCC₁₋₆ alkyl, OCO C₁₋₆ alkyl, O C₁₋₆ alkyl, S C₁₋₆ alkyl, SO C₁₋₆ alkyl, and SO₂ C₁₋₆ alkyl;
M is C₁₋₆ alkylene;
Y is a bond or C₁₋₆ alkylene;
Q is OH, COOH, or N(R^{b})₂,
R^{a} is H or C₁₋₆ alkyl,
R^{b} in each instance is independently H, C₁₋₆ alkyl, or C₂₋₆ alkyleneR^{c},
R^{c} in each instance is independently OH, SH, N(R^{a})₂, NR^{a}C₂₋₆ alkylene-OH, or N(C₂₋₆ alkylene-OH)₂,
wherein two R^{b} optionally link up to form a ring.

2. The method of claim 1, wherein the compound is of formula I-a, 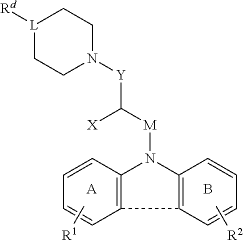
L is O, S, N or CH;
R^{d} is H, C₁₋₆ alkyl, or C₁₋₆ alkylene —OH, provided that when L is O or S, R^{d} is void.

3. The method of claim 1, wherein the compound is of formula I-b, 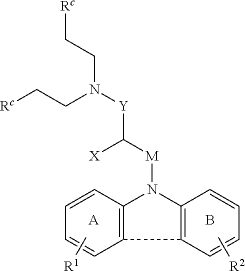
R^{c} is OH, SH, NR^{a}C₂₋₆ alkylene-OH, or N(C₂₋₆ alkylene-OH)₂.

4. The method of claim 1, wherein the compound is of formula I-c, 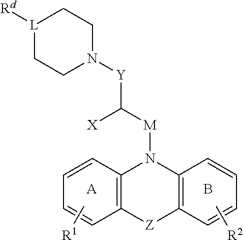
Z is O, S, NR^{a}, C(R^{a})₂, S(O)₂, or C(O);
L is O, S, N or CH;
R^{d} is H, C₁₋₆ alkyl, or C₁₋₆ alkylene —OH)₂, Provided that when L is O or S, R^{d} is void.

5. The method of claim 1, wherein the compound is of formula I-d, 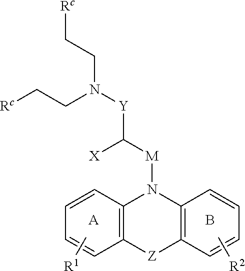
Z is O, S, NR^{a}, C(R^{a})₂, S(O)₂, or C(O);
R^{c} is OH, SH, NR^{a}C₂₋₆ alkylene-OH, or N(C₂₋₆ alkylene-OH)₂.

6. The method of claim 1, wherein the compound is selected from the group consisting of

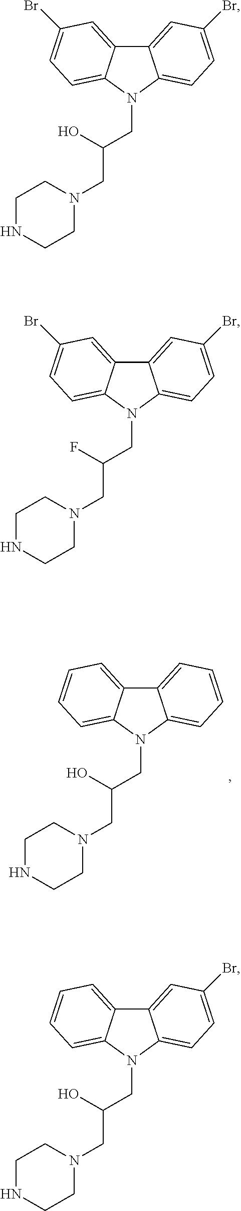 [SMILES: OC(CN1CCNCC1)Cn1c2ccc(Br)cc2c2cc(Br)ccc21 ; FC(CN1CCNCC1)Cn1c2ccc(Br)cc2c2cc(Br)ccc21 ; OC(CN1CCNCC1)Cn1c2ccccc2c2ccccc21 ; OC(CN1CCNCC1)Cn1c2ccccc2c2cc(Br)ccc21]

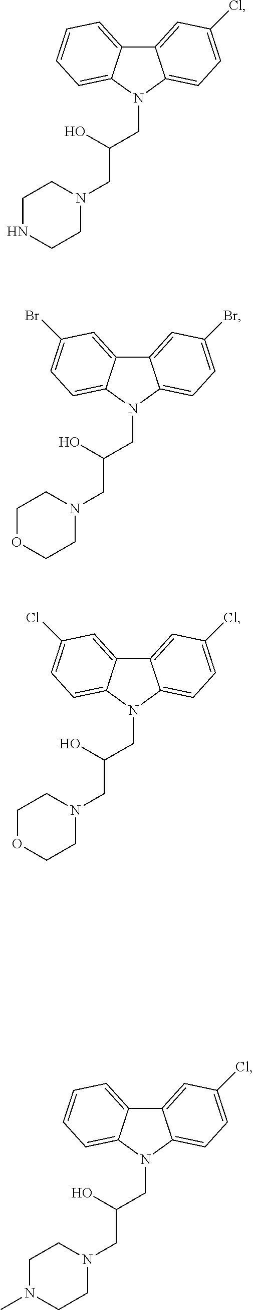 [SMILES: OC(CN1CCNCC1)Cn1c2ccccc2c2cc(Cl)ccc21 ; OC(CN1CCOCC1)Cn1c2ccc(Br)cc2c2cc(Br)ccc21 ; OC(CN1CCOCC1)Cn1c2ccc(Cl)cc2c2cc(Cl)ccc21 ; CN1CCN(CC(O)Cn2c3ccccc3c3cc(Cl)ccc32)CC1]

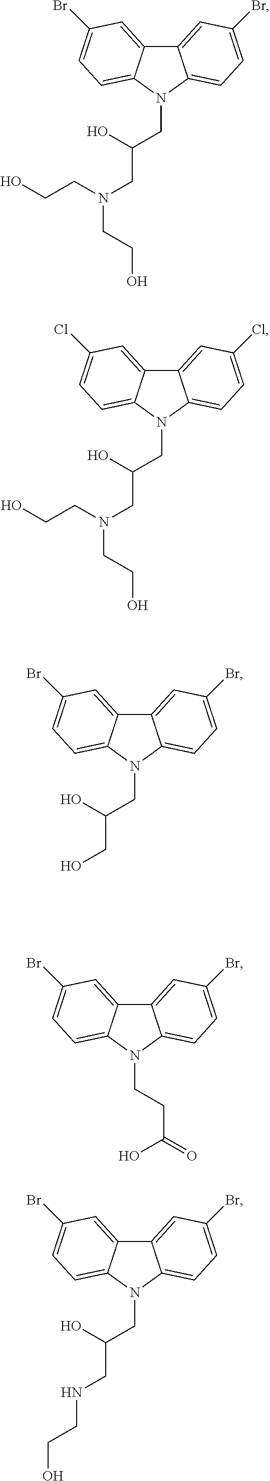 [SMILES: OCCN(CCO)CC(O)Cn1c2ccc(Br)cc2c2cc(Br)ccc21 ; OCCN(CCO)CC(O)Cn1c2ccc(Cl)cc2c2cc(Cl)ccc21 ; OCC(O)Cn1c2ccc(Br)cc2c2cc(Br)ccc21 ; O=C(O)CCn1c2ccc(Br)cc2c2cc(Br)ccc21 ; OCCNCC(O)Cn1c2ccc(Br)cc2c2cc(Br)ccc21]

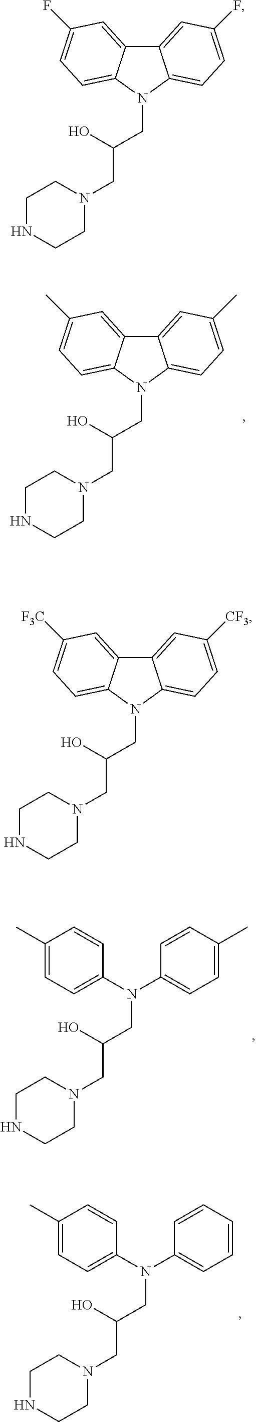 [SMILES: OC(CN1CCNCC1)Cn1c2ccc(F)cc2c2cc(F)ccc21 ; Cc1ccc2c(c1)c1cc(C)ccc1n2CC(O)CN1CCNCC1 ; OC(CN1CCNCC1)Cn1c2ccc(C(F)(F)F)cc2c2cc(C(F)(F)F)ccc21 ; Cc1ccc(N(CC(O)CN2CCNCC2)c2ccc(C)cc2)cc1 ; Cc1ccc(N(CC(O)CN2CCNCC2)c2ccccc2)cc1]

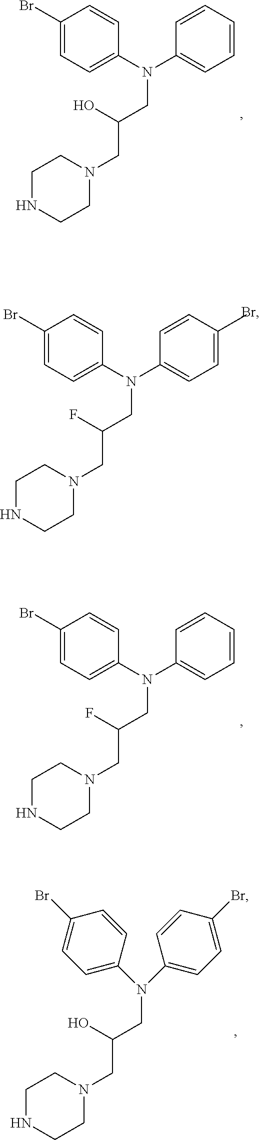

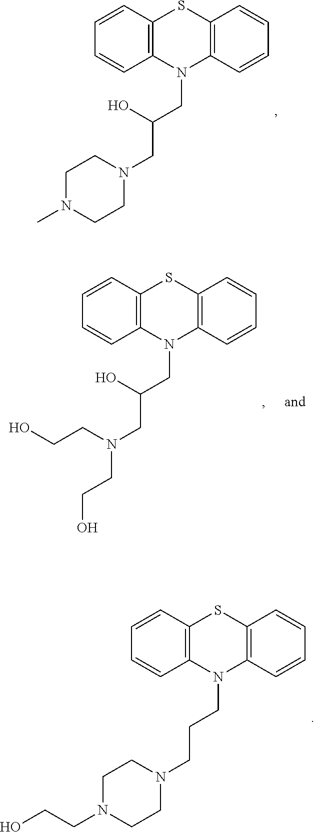 [SMILES: CN1CCN(CC(O)CN2c3ccccc3Sc3ccccc32)CC1 ; OCCN(CCO)CC(O)CN1c2ccccc2Sc2ccccc21 ; OCCN1CCN(CCCN2c3ccccc3Sc3ccccc32)CC1]

7. The method of claim 1, wherein Z is void and there is no bond between A ring and B ring.

8. The method of claim 1, wherein more than 60% of the biological material remain viable after 14 days.

9. The method of claim 1, wherein the effective amount is selected such that the biological material remains substantially viable for the period of at least about 72 hours.

10. The method of claim 1, wherein the effective amount is selected such that the biological material remains substantially viable for the period of at least about 14 days.

11. The method of claim 1, wherein the biological material is maintained in the composition at a temperature ranging from about −30° C. to about 37° C.

12. The method of claim 1, wherein the biological material is maintained in the composition at a temperature ranging from −20° C. to 5° C.

13. The method of claim 1, wherein the biological material is harvested after a preceding period of warm ischemic injury, wherein the preceding period ranges from about 2 hours to about 10 hours.

14. The method of claim 1, wherein the biological material comprises a population of mammalian cells, a mammalian tissue, or a mammalian organ.

15. The method of claim 1, wherein the biological material is selected from the group consisting of heart, lung, liver, kidney, spleen, stomach, intestine, pancreas, eye, bone, bone marrow, cochlea, and testis.

16. The method of claim 1, wherein the concentration of the compound ranges from about 1 to about 20 μg/mL in the composition.

17. The method of claim 1, further comprising, prior to the period of contact, flushing the biological material with the compound of Formula I or the pharmaceutically acceptable salt thereof.

18. A method of inhibiting cell death in a biological material ex vivo for a period of time, comprising contacting the biological sample during the period with an effective amount of a compound of Formula I or a pharmaceutically acceptable salt thereof: 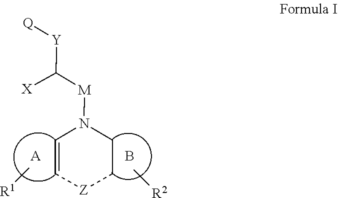
wherein
A is phenyl or a 6-membered heteroaromatic ring having 1, 2 or 3 atoms in the heteroaromatic ring;
B is phenyl, a 6-membered heteroaromatic ring having 1, 2 or 3 heteroatoms in the heteroaromatic ring, or a 6-membered aliphatic ring wherein optionally 1, 2 or 3 ring carbons are replaced with heteroatoms;
Z is void, a bond, O, S, NR^{a}, C(R^{a})₂, S(O)₂ or C(O);
the dashed line between A and B indicates an optional bond;
R¹ and R² are independently selected from the group consisting of H, C₁₋₆ alkyl, F, Cl, Br, I, CN, NO₂, N(R^{a})₂, OC₁₋₆ alkyl, CF₃, COOH, COOC₁₋₆ alkyl, OC(C═O)C₁₋₆ alkyl, OC₁₋₆ alkyl, (C═O) C₁₋₆ alkyl, C₁₋₆ alkylene-OH, SC₁₋₆ alkyl, SOC₁₋₆ alkyl, and SO₂C₁₋₆ alkyl;
X is H, F, Cl, Br, I, CN, SH, NO₂, N(R^{a})₂, OR^{a}, CF₃, COOH, C₁₋₆ alkyl, COO C₁₋₆ alkyl, OCC₁₋₆ alkyl, OCO C₁₋₆ alkyl, O C₁₋₆ alkyl, S C₁₋₆ alkyl, SO C₁₋₆ alkyl, and SO₂ C₁₋₆ alkyl;
X is H, F, Cl, Br, I, CN, SH, NO₂, N(R^{a})₂, OR^{a}, CF₃, COOH, C₁₋₆ alkyl, COO C₁₋₆ alkyl, OCC₁₋₆ alkyl, OCO C₁₋₆ alkyl, O C₁₋₆ alkyl, S C₁₋₆ alkyl, SO C₁₋₆ alkyl, and SO₂ C₁₋₆ alkyl;
M is C₁₋₆ alkylene;
Y is a bond or C₁₋₆ alkylene;
Q is OH, COOH, or N(R^{b})₂,
R^{a} is H or C₁₋₆ alkyl,
R^{b} in each instance is independently H, C₁₋₆ alkyl, or C₂₋₆ alkyleneR^{c},
R^{c} in each instance is independently OH, SH, N(R^{a})₂, NR^{a}C₂₋₆ alkylene-OH, or N(C₂₋₆ alkylene-OH)₂,
wherein two R^{b} optionally link up to form a ring.

19. A kit for storing a biological material ex vivo or prolonging the viability of the biological material ex vivo, comprising
(a) a composition comprising an effective amount of a compound of Formula I or a pharmaceutically acceptable salt thereof: 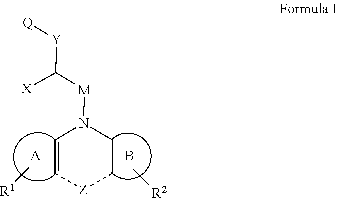
wherein
A is phenyl or a 6-membered heteroaromatic ring having 1, 2 or 3 atoms in the heteroaromatic ring;
B is phenyl, a 6-membered heteroaromatic ring having 1, 2 or 3 heteroatoms in the heteroaromatic ring, or a 6-membered aliphatic ring wherein optionally 1, 2 or 3 ring carbons are replaced with heteroatoms;
Z is void, a bond, O, S, NR^{a}, C(R^{a})₂, S(O)₂ or C(O);
the dashed line between A and B indicates an optional bond;
R¹ and R² are independently selected from the group consisting of H, C₁₋₆ alkyl, F, Cl, Br, I, CN, NO₂, N(R^{a})₂, OC₁₋₆ alkyl, CF₃, COOH, COOC₁₋₆ alkyl, OC(C═O)C₁₋₆ alkyl, OC₁₋₆ alkyl, (C═O) C₁₋₆ alkyl, C₁₋₆ alkylene-OH, SC₁₋₆ alkyl, SOC₁₋₆ alkyl, and SO₂C₁₋₆ alkyl;
X is H, F, Cl, Br, I, CN, SH, NO₂, N(R^{a})₂, OR^{a}, CF₃, COOH, C₁₋₆ alkyl, COO C₁₋₆ alkyl, OCC₁₋₆ alkyl, OCO C₁₋₆ alkyl, O C₁₋₆ alkyl, S C₁₋₆ alkyl, SO C₁₋₆ alkyl, and SO₂ C₁₋₆ alkyl;
X is H, F, Cl, Br, I, CN, SH, NO₂, N(R^{a})₂, OR^{a}, CF₃, COOH, C₁₋₆ alkyl, COO C₁₋₆ alkyl, OCC₁₋₆ alkyl, OCO C₁₋₆ alkyl, O C₁₋₆ alkyl, S C₁₋₆ alkyl, SO C₁₋₆ alkyl, and SO₂ C₁₋₆ alkyl;
M is C₁₋₆ alkylene;
Y is a bond or C₁₋₆ alkylene;
Q is OH, COOH, or N(R^{b})₂,
R^{a} is H or C₁₋₆ alkyl,
R^{b} in each instance is independently H, C₁₋₆ alkyl, or C₂₋₆ alkyleneR^{c},
R^{c} in each instance is independently OH, SH, N(R^{a})₂, NR^{a}C₂₋₆ alkylene-OH, or N(C₂₋₆ alkylene-OH)₂,
wherein two R^{b} optionally link up to form a ring;
(b) a manual on the use of the kit for storing the biological material; and
(c) optionally one or more of a biological buffer, medium, tissue storage buffer and organ transport solution.

20. The kit of claim 19, further comprising a container configured for storing the biological material, wherein the biological material is an organ selected from the group consisting of cochlea, testis, ovary, stomach, lung, heart, liver, pancreas, kidney, intestine, and eye.
